(19) United States
(12) Patent Application Publication
(10) Pub. No.: US 2021/0401674 A1
(43) Pub. Date: Dec. 30, 2021

(54) MEDICINE VENDING MACHINE WITH CLASSIFICATION FUNCTION

(71) Applicant: Xi'an Space Engine Company Limited, Xi'an (CN)

(72) Inventors: Xinfeng GUO, Xi'an (CN); Zhijun ZHOU, Xi'an (CN); Yaqing DUAN, Xi'an (CN); Yagang FENG, Xi'an (CN); Jianxin WANG, Xi'an (CN)

(21) Appl. No.: 17/470,868

(22) Filed: Sep. 9, 2021

(30) Foreign Application Priority Data
Sep. 9, 2020 (CN) 202010943721.1

(51) Int. Cl. A61J 7/00; B65G 1/08; B65G 1/137; G16H 20/13; G07F 11/42

(52) U.S. Cl. CPC A61J 7/0076; B65G 1/08; A61J 2200/70; G16H 20/13; G07F 11/42; B65G 1/137

(57) ABSTRACT

A medicine vending machine with classification function, including an electric cabinet and at least one medicine storage cabinet connected to the electric cabinet. A door of the electric cabinet is provided with a display screen, an operation area and a medicine delivery port. A medicine delivery box is provided at an interior of the electric cabinet near the medicine delivery port. A plurality of medicine cargo lanes and a medicine delivery device are provided inside the medicine storage cabinet, and a side wall of the medicine storage cabinet is provided with an isolation door. The medicine delivery device is configured to transfer the medicines stored in the medicine cargo lane to the medicine delivery box through the isolation door.

### CROSS-REFERENCE TO RELATED APPLICATIONS

This application claims the benefit of priority from Chinese Patent Application No. 202010943721.1, filed on Sep. 9, 2020. The content of the aforementioned application, including any intervening amendments thereto, is incorporated herein by reference in its entirety.

### TECHNICAL FIELD

The present disclosure relates to vending machines, and more particularly to a medicine vending machine with classification function.

### BACKGROUND

Vending machine is a machine that can automatically deliver merchandise according to the input money. Vending machines are developed for enhancing the automation degree of transactions, specifically, they are not restricted by time and space, and can facilitate saving manpower and simplifying transactions. It is a brand-new form of commercial retail, also known as a 24-hour micro supermarket. With the development of vending technology, medicine vending machines emerges on the market. However, traditional medicine vending machines cannot classify the medicines, so that the medicines are placed at random, which may cause that the medicine delivered to the user is not the desired. Moreover, the existing medicine vending machines struggle with single payment method and time-consuming medicine delivery operation.

### SUMMARY

An object of the disclosure is to provide a medicine vending machine with classification function, which has a simple structure and low maintenance cost to overcome the shortcomings of the prior art.

Technical solutions of this application are specifically described as follows.

This application provides a medicine vending machine with classification function, comprising:

an electric cabinet; and

at least one medicine storage cabinet connected to the electric cabinet;

wherein a door of the electric cabinet is provided with a display screen, an operation area and a medicine delivery port; a medicine delivery box is provided at an interior of the electric cabinet near the medicine delivery port;

a plurality of medicine cargo passages and a medicine delivery device are provided inside each of the at least one medicine storage cabinet, and a side wall of the at least one medicine storage cabinet is provided with an isolation door; and

the medicine delivery device is configured to transfer a medicine stored in the plurality of medicine cargo lanes to the medicine delivery box through the isolation door.

In some embodiments, the medicine delivery device comprises a medicine pushing mechanism, a medicine transporting mechanism and a hopper, and the hopper is provided on the medicine transporting mechanism;

the medicine pushing mechanism is configured to push the medicine stored in the plurality of medicine cargo lanes to the hopper on the medicine transporting mechanism; and

the medicine transporting mechanism is configured to transport a medicine in the hopper to the medicine delivery box in the electrical cabinet through the isolation door.

In some embodiments, the hopper is provided with a first detection device to detect whether the medicine in the hopper is a target medicine.

In some embodiments, a surface of the first detection device is coated with a coating that is capable of identifying a type of the medicine in the hopper.

In some embodiments, the medicine transporting mechanism comprises a first horizontal module, a second horizontal module, and a vertical module slidably connected to the first horizontal module and the second horizontal module;

the first horizontal module and the second horizontal module are configured to control the horizontal movement of the hopper, and the vertical module is configured to control the vertical movement of the hopper;

a sliding block is provided at an outer wall of a side of the hopper; the sliding block is provided with a through hole; and the sliding block is configured to slidably connect the hopper to the vertical module through the through hole.

In some embodiments, a bottom of the hopper is provided with a first electric push rod for driving the hopper to open and close.

In some embodiments, the hopper is provided with a vacuum chuck mechanism and a drive module; the drive module is configured to drive the vacuum chuck mechanism to move horizontally; and the vacuum chuck mechanism is configured to hold and transfer the medicine in the medicine cargo lanes into the hopper.

In some embodiments, the hopper is provided with a second detection device to detect whether the medicine in the medicine cargo lanes falls into the hopper.

In some embodiments, the medicine delivery box is provided on an inner wall of the door of the electrical cabinet, and an inclined guide plate is provided at a position on a side inner wall of the electrical cabinet corresponding to the isolation door; and

when the door of the electrical cabinet is closed, the inclined guide plate is positioned above the medicine delivery box so as to guide the medicine into the medicine delivery box.

In some embodiments, a medicine collection box is provided below the medicine delivery box, a third detection device is provided in the medicine delivery box, and a second electric push rod is provided at the bottom of the medicine delivery box;

when the third detection device detects that there are medicines remaining in the medicine delivery box, the second electric push rod drives the bottom of the medicine delivery box to open to allow the medicines to fall into the medicine collection box.

Compared to the prior art, the disclosure has the following technical effects.

In the medicine vending machine of the disclosure, the medicine storage cabinet is provided with a plurality of medicine cargo lanes, so that different types of medicines can be respectively stored in different medicine cargo lanes to achieve the classification of the medicines. As a consequence, the medicine delivery device can quickly and accurately take and deliver the medicine selected by the user, shortening the transaction time.

### BRIEF DESCRIPTION OF THE DRAWINGS

FIG. 1 is a schematic diagram of a structure of a medicine vending machine with classification function according to an embodiment of the present disclosure.

FIG. 2 is a schematic diagram of a structure of an electrical cabinet of the medicine vending machine according to an embodiment of the present disclosure.

FIG. 3 is a schematic diagram of a structure of a medicine transporting mechanism of the medicine vending machine according to an embodiment of the present disclosure

FIG. 4 is a schematic diagram of a hopper of the medicine vending machine according to an embodiment of the present disclosure.

FIG. 5 is a schematic diagram of a medicine pushing mechanism of the medicine vending machine according to an embodiment of the present disclosure.

In the drawings, 1, electrical cabinet; 2, medicine storage cabinet; 11, display screen; 12, operation area; 13, medicine delivery port; 14, medicine delivery box; 15, inclined guide plate; 16, medicine collection box; 17, second electric push rod; 21, medicine cargo lane; 22, medicine delivery device; 23, isolation door; 221, medicine push mechanism; 2211, push plate; 2212, medicine delivery module; 222, medicine transporting mechanism; 2221, first horizontal module; 2222, second horizontal module; 2223, vertical module; 2224, sliding block; 223, hopper; 2231, first detection device; 2232, first electric push rod; 2233, vacuum chuck mechanism; 2234, drive module; 2235, second detection device; and 231, third electric push rod.

### DETAILED DESCRIPTION OF EMBODIMENTS

The disclosure will be described in detail below with reference to the embodiments and accompanying drawings. It should be noted that the embodiments are merely illustrative of the disclosure, and are not intended to limit the disclosure. Unless otherwise specified, the same number in different drawings indicates the same or similar elements.

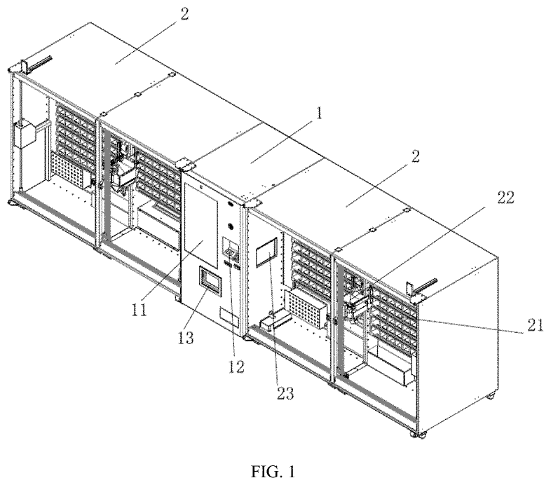

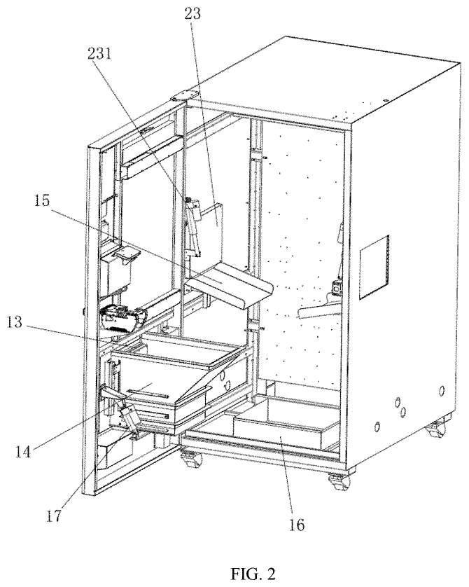

As shown in an embodiment in FIGS. 1-2, a medicine vending machine with classification function includes an electrical cabinet 1 and at least one medicine storage cabinet 2 connected to the electrical cabinet 1.

A door of the electrical cabinet 1 is provided with a display screen 11, an operation area 12 and a medicine delivery port 13, and a medicine delivery box 14 is provided at an interior of the electrical cabinet 1 near the medicine delivery port 13.

The medicine storage cabinet 2 is provided with a plurality of medicine cargo lanes 21 and a medicine delivery device 22, and a side wall of the medicine storage cabinet 2 is provided with an isolation door 23. A third electric push rod 231 is provided at the isolation door 23.

The medicine delivery device 22 can transfer the medicines stored in the medicine cargo lane 21 to the medicine delivery box 14 in the electrical cabinet 1 through the isolation door 23.

In an embodiment, the door of the electrical cabinet is also provided with a card reader to read social security card or bank card of the user, broadening the payment methods.

It should be understood that the operation area of the present disclosure can not only allow the user to select the required medicine, but also display a QR code for payment, diversifying the payment method.

It should be further noted that the display screen of the present disclosure can display advertisements about medicines or common sense of life.

The payment interface appears after the user selects the medicine through the operation area, and the payment can be made by WeChat pay, Alipay, bank card, social security card or cash. When the payment is successful, the medicine delivery device 22 will take the medicine selected by the user and send it to the medicine delivery box 14 through the isolation door 23 so that the user can take the selected medicine through the medicine delivery port.

The medicine transportation process in this disclosure is described as follows.

After the user selects a medicine through the operation area, the operation area will send a medicine delivery signal to the control system in the electrical cabinet 1, and then the control system receives the signal and controls the medicine delivery device to deliver the medicine selected by the user.

In the medicine vending machine of the disclosure, a plurality of medicine cargo lanes are provided in the medicine storage cabinet, so that different types of medicines can be respectively stored in different lanes to achieve the classification of the medicines. As a consequence, the medicine delivery device can quickly and accurately take the medicine selected by the user, shortening the transaction time.

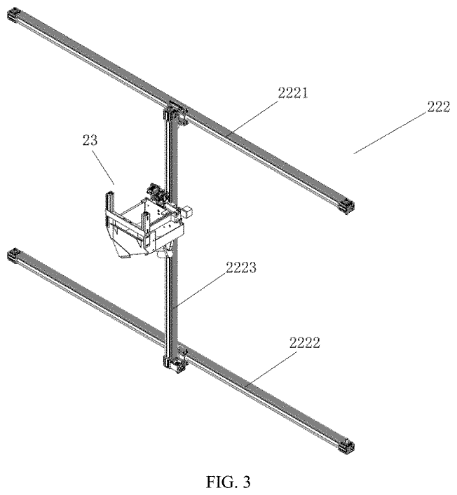

As shown in FIGS. 3-5, the medicine delivery device 22 includes a medicine pushing mechanism 221, a medicine transporting mechanism 222, and a hopper 223 provided on the medicine transporting mechanism 22.

The medicine pushing mechanism 221 is configured to push the medicines stored in the medicine cargo lanes 21 to the hopper 223 on the medicine transporting mechanism 222.

The medicine transporting mechanism 222 is configured to transfer the medicine in the hopper 223 to the medicine delivery box 14 in the electrical cabinet 1 through the isolation door 23.

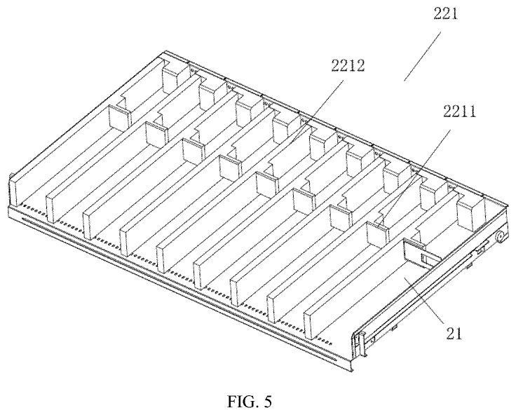

Specifically, as shown in FIG. 5, the medicine pushing mechanism 221 includes a push plate 2211 and a medicine delivery module 2212 that drives the push plate 2211 to move back and forth.

It should be understood that, in this embodiment, the medicines are placed in front of the push plate 2211 to allow the push plate to push the medicines. When the medicine is push by the push plate 2211 to the end of the medicine cargo lane 21, the medicine delivery module 2212 is continuously controlled to push the push plate 2211 to allow the medicine selected by the user to fall into the hopper 223.

It should be noted that, in this embodiment, the medicine cargo lanes are respectively provided with a corresponding medicine pushing mechanism 221. In addition, the traveling distance of the medicine pushing mechanism 221 is determined by the number of medicines selected by the user. For example, when the number of medicine A selected by the user is 1, the medicine delivery module 2212 will push the push plate 2211 to travel by 1 unit. When the number of medicine A selected by the user is 2, the medicine delivery module 2212 will push the push plate 2211 to travel by 2 units, and so on, when the number of medicine A selected by the user is n, the medicine delivery module 2212 will push the push plate to travel by n units.

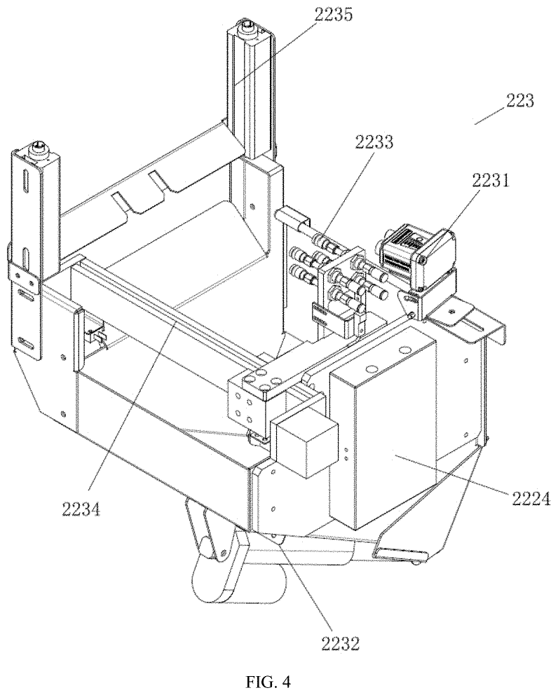

In an embodiment shown in FIG. 4, a first detection device 2231 is provided on the hopper 223 to detect whether the medicine in the hopper is the target medicine.

The arrangement of the first detection device 2231 on the hopper 223 facilitates determining whether the medicine transferred by the medicine delivery module 2212 is the medicine selected by the user.

In an embodiment, the first detection device is an infrared or ultraviolet scanning module to scan the bar code on the medicine, so as to verify whether the medicine transferred by the medicine delivery device 22 is the medicine selected by the user.

In an embodiment, the surface of the first detection device is coated with a coating that can identify the type of medicine.

In this embodiment, the coating that can identify the type of medicine is coated on the surface of the first detection device to further verify whether the medicine transferred by the medicine delivery device 22 is the medicine selected by the user.

As shown in FIGS. 3-4, the medicine transmission mechanism 222 includes a first horizontal module 2221, a second horizontal module 2222, and a vertical module 2223 slidably connected to the first horizontal module 2221 and the second horizontal module 2222.

The first horizontal module 2221 and the second horizontal module 2222 are configured to control the horizontal movement of the hopper 223, and the vertical module 2223 is configured to control the vertical movement of the hopper 223.

An outer wall of one side of the hopper 223 is provided with a sliding block 2224, and the sliding block 2224 is provided with a through hole. The sliding block 2224 is configured to slidably connect the hopper 223 to the vertical module 2223 through the through hole.

In this embodiment, the first horizontal module 2221, the second horizontal module 2222 and the vertical module 2223 are configured to horizontally and vertically move the hopper 223.

As shown in FIG. 4, the bottom of the hopper 223 is provided with a first electric push rod 2232 for driving the hopper 223 to open and close.

In this embodiment, when the medicine delivery device 22 transfers the medicine to the isolation door 23, the first electric push rod 2232 drives the hopper 223 to open, so that the medicine falls into the medicine delivery box 14 through the isolation door 23.

As shown in FIG. 4, the hopper 223 is further provided with a vacuum chuck mechanism 2233 and a drive module 2234 which can drive the vacuum chuck mechanism 2233 to move horizontally. The vacuum chuck mechanism 2233 is configured to hold and transfer the medicines in the medicine cargo lane 21 to the hopper 223.

In this embodiment, the hopper 223 is provided with the vacuum chuck mechanism 2233 and the drive module 2234 which can drive the vacuum chuck mechanism 2233 to move horizontally, such that when the medicine pushing mechanism 221 breaks down, the medicine can be held by the vacuum chuck mechanism 2233 and transferred to the hopper 223.

As shown in FIG. 4, the hopper 223 is provided with a second detection device 2235 to detect whether the medicine in the medicine cargo lane 21 falls into the hopper 223.

As shown in FIG. 2, the medicine delivery box 14 is provided on the inner wall of the door of the electrical cabinet 1, and an inclined guide plate 15 is provided at a position on a side inner wall of the electrical cabinet 1 corresponding to the isolation door 23.

When the door of the electrical cabinet 1 is closed, the inclined guide plate 15 is positioned above the medicine delivery box 14 so as to guide the medicine into the medicine delivery box 14.

In this embodiment, the inclined guide plate 15 is provided at a position on the side inner wall of the electrical cabinet 1 corresponding to the isolation door 23 to accurately and quickly pour the medicine selected by the user into the medicine delivery box 14.

As shown in FIG. 2, a medicine collection box 16 is provided below the medicine delivery box 14, and the third detection device is provided in the medicine taking box 14. A second electric push rod 17 is provided at the bottom of the medicine delivery box 14.

When the third detection device detects that there are medicines remaining in the medicine delivery box 14, the second electric push rod 17 drives the bottom of the medicine delivery box 14 to open, such that the medicines can fall into the medicine collection box 16.

In this embodiment, the medicine collection box 16 is provided below the medicine delivery box 14; the third detecting device is provided in the medicine delivery box 14; and the second electric push rod 17 is provided at the bottom of the medicine delivery box 14. After the third detecting device that the medicines selected by the user have not been completely taken from the medicine delivery box 14, the third detection device will send a prompt signal to the control system, and the control system will control the second electric push rod 17 to open the bottom of the medicine delivery box 14, so that medicines that the previous user does not take away fall into the medicine collection box 16, thereby preventing the next user from taking the medicines purchased by the previous user.

In an embodiment, the medicine delivery port 13 is configured as an automatic lifting door, and the automatic lifting door is provided with an anti-pinch device.

In an embodiment, the electric cabinet 1 and the medicine storage cabinet 2 of the medicine vending machine are respectively provided with universal wheels at the bottom to facilitate the movement of the medicine vending machine.

It should be noted that described above are merely preferred embodiments of the disclosure, which are not intended to limit the disclosure. It should be understood that any modifications, replacements and changes made by those skilled in the art without departing from the spirit of the disclosure shall fall within the scope of the disclosure defined by the appended claims.

What is claimed is:

1. A medicine vending machine with classification function, comprising:
an electric cabinet; and
at least one medicine storage cabinet connected to the electric cabinet;
wherein a door of the electric cabinet is provided with a display screen, an operation area and a medicine delivery port; a medicine delivery box is provided at an interior of the electric cabinet near the medicine delivery port;
a plurality of medicine cargo lanes and a medicine delivery device are provided inside each of the at least one medicine storage cabinet, and a side wall of the at least one medicine storage cabinet is provided with an isolation door; and
the medicine delivery device is configured to transfer a medicine stored in the plurality of medicine cargo lanes to the medicine delivery box through the isolation door.

2. The medicine vending machine of claim 1, wherein the medicine delivery device comprises a medicine pushing mechanism, a medicine transporting mechanism, and a hopper, and the hopper is provided on the medicine transporting mechanism;
the medicine pushing mechanism is configured to push the medicine stored in the plurality of medicine cargo lanes to the hopper on the medicine transporting mechanism; and
the medicine transporting mechanism is configured to transport a medicine in the hopper to the medicine delivery box in the electrical cabinet through the isolation door.

3. The medicine vending machine of claim 2, wherein the hopper is provided with a first detection device, and the first detection device is configured to detect whether the medicine in the hopper is a target medicine.

4. The medicine vending machine of claim 3, wherein a surface of the first detection device is coated with a coating that is capable of identifying a type of the medicine in the hopper.

5. The medicine vending machine of claim 2, wherein the medicine transporting mechanism comprises a first horizontal module, a second horizontal module and a vertical module slidably connected to the first horizontal module and the second horizontal module;
the first horizontal module and the second horizontal module are configured to control horizontal movement of the hopper, and the vertical module is configured to control vertical movement of the hopper; and
a sliding block is provided at an outer wall of a side of the hopper; the sliding block is provided with a through hole; and the sliding block is configured to slidably connect the hopper to the vertical module through the through hole.

6. The medicine vending machine of claim 2, wherein a bottom of the hopper is provided with a first electric push rod for driving the hopper to open and close.

7. The medicine vending machine of claim 3, wherein a bottom of the hopper is provided with a first electric push rod for driving the hopper to open and close.

8. The medicine vending machine of claim 2, wherein the hopper is further provided with a vacuum chuck mechanism and a drive module; the drive module is configured to drive the vacuum chuck mechanism to move horizontally; and the vacuum chuck mechanism is configured to hold and transfer the medicine in the plurality of medicine cargo lanes into the hopper.

9. The medicine vending machine of claim 3, wherein the hopper is further provided with a second detection device, and the second detection device is configured to detect whether the medicine in the plurality of medicine cargo lanes fall into the hopper.

10. The medicine vending machine of claim 7, wherein the hopper is further provided with a second detection device, and the second detection device is configured to detect whether the medicine in the plurality of medicine cargo lanes fall into the hopper.

11. The medicine vending machine of claim 1, wherein the medicine delivery box is provided on an inner wall of the door of the electrical cabinet, and an inclined guide plate is provided at a position on a side inner wall of the electrical cabinet corresponding to the isolation door; and
when the door of the electrical cabinet is closed, the inclined guide plate is located above the medicine delivery box so as to guide the medicine into the medicine delivery box.

12. The medicine vending machine of claim 1, wherein a medicine collection box is provided below the medicine delivery box; a detection device is provided in the medicine delivery box; and an electric push rod is provided at a bottom of the medicine delivery box; and
when the detection device detects that there are medicines remaining in the medicine delivery box, the electric push rod drives to open the bottom of the medicine delivery box to allow the medicines to fall into the medicine collection box.

13. The medicine vending machine of claim 10, wherein a medicine collection box is provided below the medicine delivery box; a third detection device is provided in the medicine delivery box; and a second electric push rod is provided at the bottom of the medicine delivery box; and
when the third detection device detects that there are medicines remaining in the medicine delivery box, the second electric push rod drives the bottom of the medicine delivery box to open to allow the medicines to fall into the medicine collection box.
